(19) United States
(12) Patent Application Publication
(10) Pub. No.: US 2022/0241562 A1
(43) Pub. Date: Aug. 4, 2022

(54) Sheath Stabilizer, Sheath-And-Stabilizer Assembly, And Methods Thereof

(71) Applicant: Becton, Dickinson and Company, Franklin Lakes, NJ (US)

(72) Inventors: Peng Yi, Shanghai (CN); Xiaowen Sun, Shanghai (CN); Zhixiu He, Shanghai (CN); Neville Chia, Singapore (SG)

(21) Appl. No.: 17/612,196

(22) PCT Filed: May 24, 2019
(86) PCT No.: PCT/CN19/88347

§ 371 Date: Nov. 17, 2021

(51) Int. Cl. A61M 25/06; A61M 25/02

(52) U.S. Cl. CPC A61M 25/0612; A61M 2005/1581; A61M 25/02

(57) ABSTRACT

A sheath stabilizer can include a housing, a clamp in the housing, and an adhesive pad disposed on a base of the housing. The housing can include a sheath passageway longitudinally extending through the housing and the base oblique to the sheath passageway. The clamp can be located in a proximal-end portion of the housing around the sheath passageway. The adhesive pad can be disposed on the base and can be configured for adhering the sheath stabilizer to at least a patients skin. The sheath stabilizer can be part of a sheath-and-stabilizer assembly, which can include a sheath configured for percutaneous access. Methods for the sheath stabilizer are described.

### BACKGROUND

In a healthy person, blood flowing from the stomach, esophagus, or intestines first flows through the liver. In an unhealthy person having, for example, liver damage, there can be blood flow-restricting blockages in the liver such that blood cannot easily flow therethrough. Such a condition is known as portal hypertension. Common causes of portal hypertension include alcohol abuse, too much iron in the liver (e.g., hemochromatosis), hepatitis B, hepatitis C, or blood clots in a vein that flows from the liver to the heart. When portal hypertension occurs, the blood flow-restricting blockages can elevate pressure in the portal vein causing it to rupture and seriously bleed. A person with portal hypertension can also have bleeding from the veins of the stomach, esophagus, or intestines (e.g., variceal bleeding), a buildup of fluid in the belly (e.g., ascites), or a buildup of fluid in the chest (e.g., hydrothorax).

Portal hypertension is often treated by way of a percutaneous procedure involving placement of a transjugular intrahepatic portosystemic shunt (“TIPS”) between the hepatic vein and the portal vein as shown in FIG. 7 to establish blood flow through the liver. During the procedure, an introducer sheath is positioned in the hepatic vein and used to introduce both a stiffening cannula and a needle for a subsequent needle throw from the hepatic vein to the portal vein. During such a needle throw, a clinician typically uses one hand to hold the stiffening cannula and the other hand to throw the needle. In order to maintain the position of the sheath during the needle throw, an assistant is typically needed to hold the sheath in position; however, an assistant is not always available. In addition, needing an assistant to hold the sheath in position during the needle throw adds complexity to the procedure.

Disclosed herein is a sheath stabilizer, a sheath-and-stabilizer assembly, and methods thereof that address at least the forgoing shortcomings.

### SUMMARY

Disclosed herein is a sheath stabilizer including, in some embodiments, a housing, a clamp in the housing, and an adhesive pad disposed on a base of the housing. The housing includes a sheath passageway longitudinally extending through the housing and the base oblique to the sheath passageway. The clamp is in a proximal-end portion of the housing around the sheath passageway. The adhesive pad disposed on the base is configured for adhering the sheath stabilizer to at least a patient's skin.

In some embodiments, the clamp includes a pair of opposing buttons disposed in buttonholes of the housing. The buttons are configured to open the clamp when the buttons are pressed into the housing toward each other.

In some embodiments, the clamp includes a pair of opposing clamp pieces along the sheath passageway. Each clamp piece of the clamp pieces is coupled by a strut to a button of the buttons on an opposite side of the housing.

In some embodiments, each clamp piece of the clamp pieces is integral with the button on the opposite side of the housing.

In some embodiments, each clamp piece of the clamp pieces is approximated in shape by a longitudinal cross section of an open right cylinder.

In some embodiments, each clamp piece of the clamp pieces has an elastomer-lined face facing the sheath passageway. The elastomer is structured as a pad.

In some embodiments, the clamp includes a pair of opposing compression springs orthogonal to the sheath passageway. Each spring of the springs is disposed between a back of a button of the buttons and a back of a clamp piece of the clamp pieces.

In some embodiments, the sheath passageway is configured to accept up to a 16-Fr sheath inserted therein.

In some embodiments, the adhesive pad includes a ready-to-peel backing thereon configured to expose an adhesive when the backing is peeled off the adhesive pad.

In some embodiments, the housing includes molded mirrored-imaged halves of the housing pressed or adhered together over the clamp.

Also disclosed herein is a sheath-and-stabilizer assembly including, in some embodiments, a sheath configured for percutaneous access and a sheath stabilizer. The sheath stabilizer includes a housing, a clamp in the housing, and an adhesive pad disposed on a base of the housing. The housing includes a sheath passageway longitudinally extending through the housing and the base oblique to the sheath passageway. The clamp is in a proximal-end portion of the housing around the sheath passageway. The adhesive pad disposed on the base is configured for adhering the sheath stabilizer to at least a patient's skin.

In some embodiments, the clamp includes a pair of opposing buttons disposed in buttonholes of the housing. The buttons are configured to open the clamp when the buttons are pressed into the housing toward each other. The buttons are configured to close the clamp when the buttons are released and allowed to move away from each other.

In some embodiments, the clamp includes a pair of opposing clamp pieces along the sheath passageway. Each clamp piece of the clamp pieces is integral with a strut-connected button of the buttons on an opposite side of the housing.

In some embodiments, the clamp includes a pair of opposing compression springs orthogonal to the sheath passageway. Each spring of the springs is disposed between a back of a button of the buttons and a back of a clamp piece of the clamp pieces.

In some embodiments, the sheath passageway is configured to accept up to a 16-Fr sheath inserted therein.

Also disclosed herein is a method for a sheath stabilizer including, in some embodiments, opening a clamp of the sheath stabilizer by pressing a pair of opposing buttons toward each other, the buttons disposed in buttonholes in a proximal-end portion of a housing of the sheath stabilizer; inserting a sheath configured for percutaneous access into a sheath passageway longitudinally extending through the housing of the sheath stabilizer; inserting the sheath into a percutaneous access site; and adhering a base of the sheath stabilizer to a patient's skin, the base of the sheath stabilizer oblique to the sheath passageway such that the sheath maintains an insertion angle at the percutaneous access site.

In some embodiments, inserting the sheath into the sheath passageway of the sheath stabilizer includes positioning a proximal-end portion of the sheath in the clamp.

In some embodiments, the method further includes closing the clamp of the sheath stabilizer around the sheath by releasing the buttons and allowing the buttons to move away from each other.

In some embodiments, the method further includes removing a ready-to-peel backing from an adhesive pad disposed on the base of the sheath stabilizer to expose an adhesive before adhering the base of the sheath stabilizer to the patient's skin.

In some embodiments, inserting the sheath into the percutaneous access site includes advancing the sheath to the hepatic vein.

In some embodiments, the method further includes inserting a dilator into the sheath if the sheath is not provided as a sheath-and-dilator assembly; and dilating the percutaneous access site with the dilator when inserting the sheath into the percutaneous access site.

These and other features of the concepts provided herein will become more apparent to those of skill in the art in view of the accompanying drawings and following description, which disclose particular embodiments of such concepts in greater detail.

### DRAWINGS

FIG. 1 illustrates a sheath stabilizer, in accordance with some embodiments.

FIG. 2 illustrates a clamp of the sheath stabilizer, in accordance with some embodiments.

FIG. 3 illustrates a cross section of the sheath stabilizer when the clamp of the sheath stabilizer is clamped around a sheath, in accordance with some embodiments.

FIG. 4 illustrates a cross section of the sheath stabilizer when the clamp of the sheath stabilizer is not clamped around a sheath, in accordance with some embodiments.

FIG. 5 illustrates a sheath-and-stabilizer assembly, in accordance with some embodiments.

FIG. 6 illustrates inserting a sheath into the sheath stabilizer, in accordance with some embodiments.

FIG. 7 illustrates a procedure involving placement of a TIPS between a hepatic vein and a portal vein, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a sheath disclosed herein includes a portion of the sheath intended to be near a clinician when the sheath is used on a patient. Likewise, a “proximal length” of, for example, the sheath includes a length of the sheath intended to be near the clinician when the sheath is used on the patient. A “proximal end” of, for example, the sheath includes an end of the sheath intended to be near the clinician when the sheath is used on the patient. The proximal portion, the proximal-end portion, or the proximal length of the sheath can include the proximal end of the sheath; however, the proximal portion, the proximal-end portion, or the proximal length of the sheath need not include the proximal end of the sheath. That is, unless context suggests otherwise, the proximal portion, the proximal-end portion, or the proximal length of the sheath is not a terminal portion or terminal length of the sheath.

With respect to “distal,” a “distal portion” or a “distal-end portion” of, for example, a sheath disclosed herein includes a portion of the sheath intended to be near or in a patient when the sheath is used on the patient. Likewise, a “distal length” of, for example, the sheath includes a length of the sheath intended to be near or in the patient when the sheath is used on the patient. A “distal end” of, for example, the sheath includes an end of the sheath intended to be near or in the patient when the sheath is used on the patient. The distal portion, the distal-end portion, or the distal length of the sheath can include the distal end of the sheath; however, the distal portion, the distal-end portion, or the distal length of the sheath need not include the distal end of the sheath. That is, unless context suggests otherwise, the distal portion, the distal-end portion, or the distal length of the sheath is not a terminal portion or terminal length of the sheath.

Unless defined otherwise, all technical and scientific terms used herein have the same meaning as commonly understood by those of ordinary skill in the art.

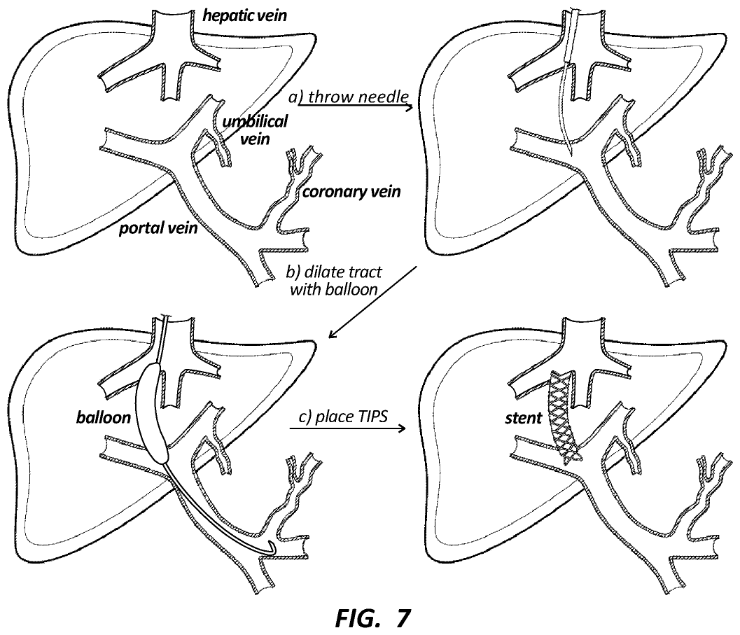

As set forth above, portal hypertension is often treated by way of a percutaneous procedure involving placement of a TIPS between the hepatic vein and the portal vein as shown in FIG. 7 to establish blood flow through the liver. During the procedure, an introducer sheath is positioned in the hepatic vein and used to introduce both a stiffening cannula and a needle for a subsequent needle throw from the hepatic vein to the portal vein. During such a needle throw, a clinician typically uses one hand to hold the stiffening cannula and the other hand to throw the needle. In order to maintain the position of the sheath during the needle throw, an assistant is typically needed to hold the sheath in position; however, an assistant is not always available. In addition, needing an assistant to hold the sheath in position during the needle throw adds complexity to the procedure.

Disclosed herein is a sheath stabilizer, a sheath-and-stabilizer assembly, and methods thereof that address at least the forgoing shortcomings. For example, in the foregoing TIPS procedure a clinician can use one hand to hold the stiffening cannula and the other hand to throw the needle while the sheath stabilizer holds the sheath in position. It should be understood the sheath stabilizer, the sheath-and-stabilizer assembly, and the methods thereof are not limited to TIPS placement. Indeed, the foregoing shortcomings in TIPS placement merely exemplify the need for the sheath stabilizer, the sheath-and-stabilizer assembly, and the methods thereof disclosed herein.

### Sheath Stabilizer

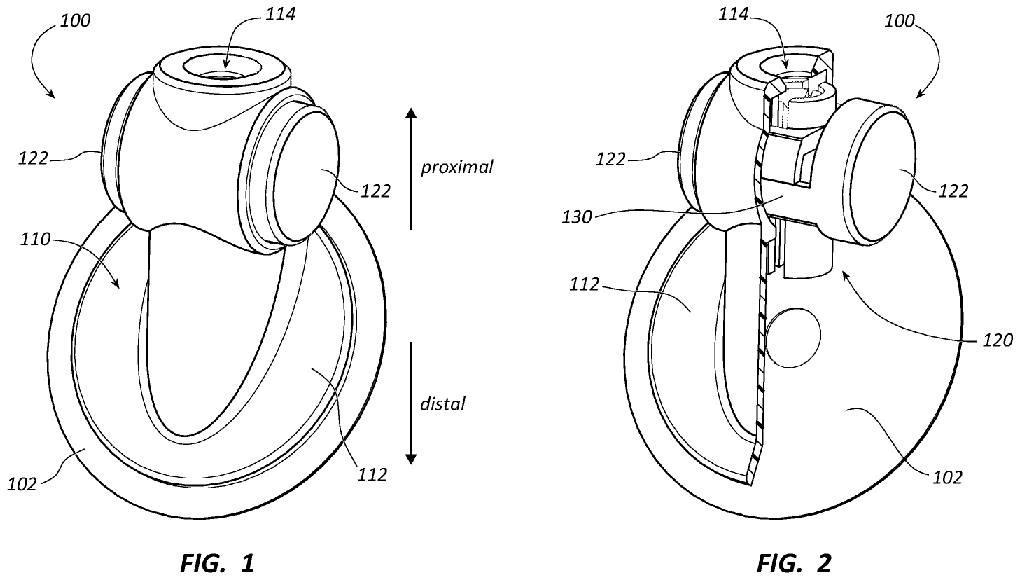

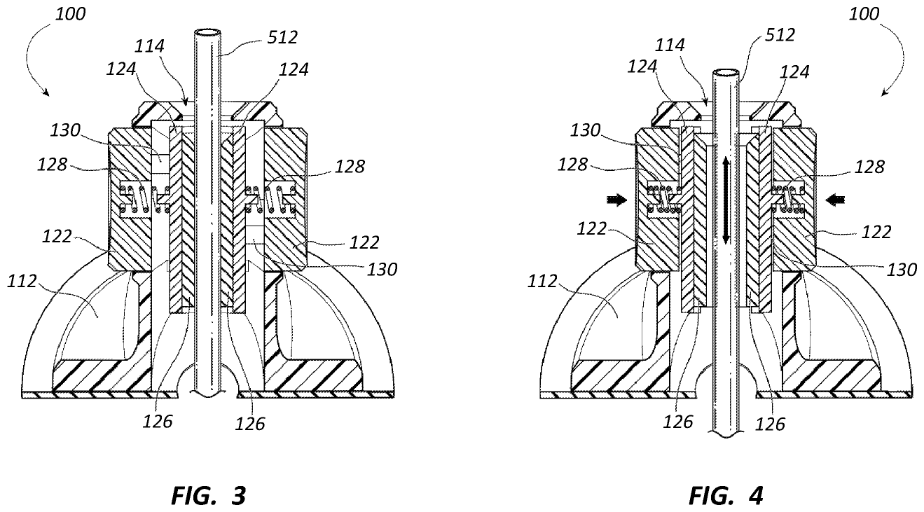

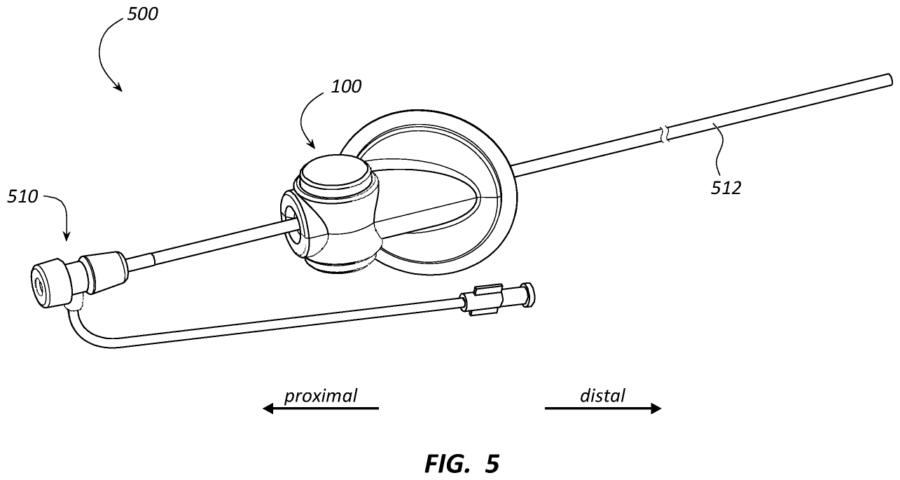

FIG. 1 illustrates a sheath stabilizer 100, in accordance with some embodiments. FIG. 2 illustrates a clamp 120 of the sheath stabilizer 100, in accordance with some embodiments. FIG. 3 illustrates a cross section of the sheath stabilizer 100 when the clamp 120 of the sheath stabilizer 100 is clamped around a sheath, in accordance with some embodiments. FIG. 4 illustrates a cross section of the sheath stabilizer 100 when the clamp 120 of the sheath stabilizer 100 is not clamped around a sheath, in accordance with some embodiments.

As shown, the sheath stabilizer 100 includes a housing 110, the clamp 120 in the housing 110, and an adhesive pad 102 disposed on a base 112 of the housing 110.

The housing 110 includes a sheath passageway 114 longitudinally extending through the housing 110 from a proximal end of the housing 110 to a distal end of the housing 110. The sheath passageway 114 is configured to accept at least a 4-Fr sheath, a 6-Fr sheath, an 8-Fr sheath, a 10-Fr sheath, a 12-Fr sheath, a 14-Fr sheath, or a 16-Fr sheath inserted therein. The sheath passageway 114 is configured to accept no more than a 16-Fr sheath, a 14-Fr sheath, a 12-Fr sheath, a 10-Fr sheath, an 8-Fr sheath, a 6-Fr sheath, or a 4-Fr sheath inserted therein. Thus, the sheath passageway 114 can be sized in accordance with the sheath needed for a particular percutaneous procedure.

The base 112 of the housing 110 is oblique to the sheath passageway 114 such that when the base is placed over a percutaneous access site of a patient, the sheath passageway 114 is near or at an acceptable insertion angle for inserting a sheath into the percutaneous access site. The base 112 can be planar or curved to match a body of the patient about one or more acceptable body locations for percutaneous access. Thus, the base 112 can be shaped in accordance with that needed for a particular percutaneous procedure.

The housing 110 can be made by molding pieces of the housing 110 and coupling the resulting molded pieces together to make the housing 110. Since the housing 110 of FIGS. 1-4 has bilateral symmetry, mirror-imaged halves of the housing 110 can be molded by, for example, injection molding with polyethylene, polycarbonate, or some other medically acceptable thermoplastic. The resulting molded halves of the housing 110 can be coupled together over the clamp 120 by pressing the molded halves together if configured with snap-together connectors, adhering the molded halves together with an adhesive, bonding the molded halves together with solvent, or a combination thereof.

The clamp 120 includes a pair of opposing buttons 122 disposed in buttonholes in a proximal-end portion of the housing 110 around the sheath passageway 114. As shown in FIG. 4, the buttons 122 are configured to open the clamp 120 when the buttons 122 are pressed into the housing 110 toward each other. As shown in FIG. 3, the buttons 122 are configured to close the clamp 120 when the buttons 122 are released and allowed to move away from each other. The sheath tube 512 of the sheath 510 of FIG. 5 is shown in FIGS. 3 and 4 to illustrate how the clamp 120 opens to accept (or release) a sheath and closes to clamp the sheath.

The clamp 120 includes a pair of opposing clamp pieces 124 along the sheath passageway 114. Each clamp piece of the clamp pieces 124 is approximated in shape by a longitudinal cross section of an open right cylinder. Such a shape for the clamp pieces 124 enables an equal clamping pressure to be applied by the clamp pieces 124 around a sheath. Each clamp piece of the clamp pieces 124 has an elastomer-lined face facing the sheath passageway 114. Such an elastomeric lining can be structured as a deformable pad 126 such as a foam or rubber pad adhered to each clamp piece of the clamp pieces 124 to cushion a sheath between the clamp pieces 124 and provide friction to hold the sheath between the clamp pieces 124.

Each button of the buttons 122 is coupled by a strut 130 to a clamp piece of the clamp pieces 124 on an opposite side of the housing 110. For example, as shown in FIGS. 3 and 4, the left-hand button of the buttons 122 is coupled by the referenced strut 130 to the right-hand clamp piece of the clamp pieces 124. Likewise, the right-hand button of the buttons 122 is coupled by a strut to the left-hand clamp piece of the clamp pieces 124. Thus, the clamp 120 is constructed from a number of interlocking pieces. When molding (e.g., injection molding with polyethylene, polycarbonate, or some other medically acceptable thermoplastic) is used to make the buttons 122, the clamp pieces 124, and the struts, each button can be molded such that it is integral with a clamp piece by way of a strut therebetween, thereby forming integral clamp pieces. Due to a two-fold rotational symmetry possessed by each integral clamp piece of the integral clamp pieces, any two integral clamp pieces interlock to form the clamp 120.

The clamp 120 includes a pair of opposing compression springs 128 orthogonal to the sheath passageway 114. Each spring of the springs 128 is disposed between a back of a button of the buttons 122 and a back of a clamp piece of the clamp pieces 124 in at least a slightly compressed state. Because compression springs are configured to resist compression, the clamp pieces 124 are forced together and the buttons 122 are forced apart in a default or resting state of the clamp 120. Again, the buttons 122 are configured to open the clamp 120 when the buttons 122 are pressed into the housing 110 toward each other, which further compresses the springs 128. When the buttons 122 are released and allowed to move away from each other, the clamp 120 returns to the default or resting state of the clamp 120, which is the state of the clamp 120 used to clamp a sheath therein.

The adhesive pad 102 is disposed on a patient-facing side of the base 112 of the sheath stabilizer 100 and held thereon by a suitable adhesive. The adhesive pad 102, in turn, includes a medically acceptable adhesive on a patient-facing side of the adhesive pad 102 configured for adhering the sheath stabilizer 100 to at least a patient's skin. Prior to use, the adhesive pad 102 includes a ready-to-peel backing thereon configured to expose the medically acceptable adhesive when the backing is peeled off the adhesive pad 102.

When such a sheath stabilizer 100 is used to stabilize a sheath during a procedure such as a TIPS procedure, any external loads applied to the sheath are attenuated by way of transmission through the sheath stabilizer 100 to an outside of the patient where the sheath stabilizer 100 is adhered, thereby reducing direct trauma to the percutaneous access site, a vessel of the patient, or the like.

### Sheath-and-Stabilizer Assembly

FIG. 5 illustrates a sheath-and-stabilizer assembly 500, in accordance with some embodiments.

As shown, the sheath-and-stabilizer assembly 500 includes a sheath 510 configured for percutaneous access and the sheath stabilizer 100. When assembled as shown, a proximal-end portion of a sheath tube 512 of the sheath 510 is clamped in the sheath passageway 114 by the clamp 120 of the sheath stabilizer 100. The sheath tube 512 can range from 4 Fr to 16 Fr as the sheath passageway 114 is configured to accept a sheath from 4 Fr to 16 Fr as set forth above.

A kit or set for a procedure such as a TIPS procedure includes the sheath-and-stabilizer assembly 500 either assembled as shown in FIG. 5 or ready to be assembled.

### Methods

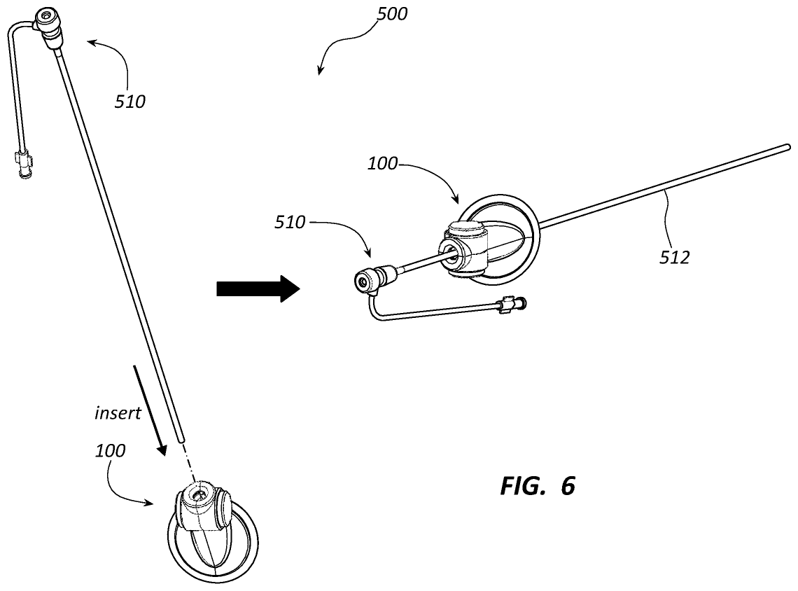

FIG. 6 illustrates inserting the sheath 510 into the sheath stabilizer 100, in accordance with some embodiments.

A method for the sheath stabilizer 100 includes opening the clamp 120 of the sheath stabilizer 100 by pressing the pair of opposing buttons 122 toward each other, the buttons 122 disposed in buttonholes in a proximal-end portion of the housing 110 of the sheath stabilizer 100; inserting the sheath tube 512 of the sheath 510 into the sheath passageway 114 longitudinally extending through the housing 110 of the sheath stabilizer 100; inserting the sheath tube 512 into a percutaneous access site; and adhering the base 112 of the sheath stabilizer 100 to a patient's skin, the base 112 of the sheath stabilizer 100 oblique to the sheath passageway 114 such that the sheath tube 512 maintains an insertion angle at the percutaneous access site. Inserting the sheath tube 512 into the sheath passageway 114 of the sheath stabilizer 100 includes positioning a proximal-end portion of the sheath tube 512 in the clamp 120.

The method further includes closing the clamp 120 of the sheath stabilizer 100 around the sheath tube 512 by releasing the buttons 122 and allowing the buttons 122 to move away from each other.

The method further includes removing a ready-to-peel backing from the adhesive pad 102 disposed on the base 112 of the sheath stabilizer 100 to expose a medically acceptable adhesive before adhering the base 112 of the sheath stabilizer 100 to the patient's skin.

When performing a procedure such as a TIPS procedure, inserting the sheath tube 512 into the percutaneous access site includes advancing a distal-end portion of the sheath 512 to the hepatic vein.

The method further includes inserting a dilator into the sheath 510 if the sheath 510 is not provided as a sheath-and-dilator assembly; and dilating the percutaneous access site with the dilator when inserting the sheath tube 512 into the percutaneous access site.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A sheath stabilizer, comprising:
a housing including a sheath passageway longitudinally extending through the housing and a base oblique to the sheath passageway;
a clamp in a proximal-end portion of the housing around the sheath passageway; and
an adhesive pad disposed on the base configured for adhering the sheath stabilizer to at least a patient's skin.

2. The sheath stabilizer of claim 1, wherein the clamp includes a pair of opposing buttons disposed in buttonholes of the housing, the buttons configured to open the clamp when the buttons are pressed into the housing toward each other.

3. The sheath stabilizer of claim 2, wherein the clamp includes a pair of opposing clamp pieces along the sheath passageway, each clamp piece of the clamp pieces coupled by a strut to a button of the buttons on an opposite side of the housing.

4. The sheath stabilizer of claim 3, wherein each clamp piece of the clamp pieces is integral with the button on the opposite side of the housing.

5. The sheath stabilizer of claim 3, wherein each clamp piece of the clamp pieces is approximated in shape by a longitudinal cross section of an open right cylinder.

6. The sheath stabilizer of claim 3, wherein each clamp piece of the clamp pieces has an elastomer-lined face facing the sheath passageway, the elastomer structured as a pad.

7. The sheath stabilizer of claim 3, wherein the clamp includes a pair of opposing compression springs orthogonal to the sheath passageway, each spring of the springs disposed between a back of a button of the buttons and a back of a clamp piece of the clamp pieces.

8. The sheath stabilizer of claim 1, wherein the sheath passageway is configured to accept up to a 16-Fr sheath inserted therein.

9. The sheath stabilizer of claim 1, wherein the adhesive pad includes a ready-to-peel backing thereon configured to expose an adhesive when the backing is peeled off the adhesive pad.

10. The sheath stabilizer of claim 1, wherein the housing includes molded mirrored-imaged halves of the housing pressed or adhered together over the clamp.

11. A sheath-and-stabilizer assembly, comprising:
a) sheath configured for percutaneous access; and
b) a sheath stabilizer including:
a housing including a sheath passageway longitudinally extending through the housing and a base oblique to the sheath passageway;
a clamp in a proximal-end portion of the housing around the sheath passageway; and
an adhesive pad disposed on the base configured for adhering the sheath stabilizer to at least a patient's skin.

12. The sheath stabilizer of claim 11, wherein the clamp includes a pair of opposing buttons disposed in buttonholes of the housing, the buttons configured to open the clamp when the buttons are pressed into the housing toward each other and close the clamp when the buttons are released and allowed to move away from each other.

13. The sheath stabilizer of claim 11, wherein the clamp includes a pair of opposing clamp pieces along the sheath passageway, each clamp piece of the clamp pieces integral with a strut-connected button of the buttons on an opposite side of the housing.

14. The sheath stabilizer of claim 13, wherein the clamp includes a pair of opposing compression springs orthogonal to the sheath passageway, each spring of the springs disposed between a back of a button of the buttons and a back of a clamp piece of the clamp pieces.

15. The sheath stabilizer of claim 11, wherein the sheath passageway is configured to accept up to a 16-Fr sheath inserted therein.

16. A method for a sheath stabilizer, comprising:
opening a clamp of the sheath stabilizer by pressing a pair of opposing buttons toward each other, the buttons disposed in buttonholes in a proximal-end portion of a housing of the sheath stabilizer;
inserting a sheath configured for percutaneous access into a sheath passageway longitudinally extending through the housing of the sheath stabilizer;
inserting the sheath into a percutaneous access site; and
adhering a base of the sheath stabilizer to a patient's skin, the base of the sheath stabilizer oblique to the sheath passageway such that the sheath maintains an insertion angle at the percutaneous access site.

17. The method of claim 16, wherein inserting the sheath into the sheath passageway of the sheath stabilizer includes positioning a proximal-end portion of the sheath in the clamp.

18. The method of claim 16, further comprising:
closing the clamp of the sheath stabilizer around the sheath by releasing the buttons and allowing the buttons to move away from each other.

19. The method of claim 16, further comprising:
removing a ready-to-peel backing from an adhesive pad disposed on the base of the sheath stabilizer to expose an adhesive before adhering the base of the sheath stabilizer to the patient's skin.

20. The method of claim 16, wherein inserting the sheath into the percutaneous access site includes advancing the sheath to the hepatic vein.

21. The method of claim 16, further comprising:
inserting a dilator into the sheath if the sheath is not provided as a sheath-and-dilator assembly; and
dilating the percutaneous access site with the dilator when inserting the sheath into the percutaneous access site.
